(19) United States
(12) Patent Application Publication
(10) Pub. No.: US 2023/0293774 A1
(43) Pub. Date: Sep. 21, 2023

(54) STENT GRAFT WITH ELASTOMERIC IMPERMEABLE LAYER

(71) Applicant: Merit Medical Systems, Inc., South Jordan, UT (US)

(72) Inventors: John Hall, Bountiful, UT (US); Wayne Mower, Bountiful, UT (US)

(21) Appl. No.: 18/183,827

(22) Filed: Mar. 14, 2023

Related U.S. Application Data
(60) Provisional application No. 63/269,366, filed on Mar. 15, 2022.

(51) Int. Cl. A61L 27/50; A61F 2/07; A61L 27/26

(52) U.S. Cl. CPC A61L 27/507; A61F 2/07; A61L 27/26; A61F 2002/072; A61F 2210/0057; A61F 2210/0076; A61F 2240/001; A61F 2250/0039

(57) ABSTRACT

Medical appliances, such as stent grafts, may be formed of a cover and a scaffolding. The cover is composed of multiple layers of polymeric materials. A luminal layer is formed of rotational spun fibers of polytetrafluoroethylene. An abluminal layer is formed of expanded polytetrafluoroethylene. The scaffolding is disposed between the luminal layer and the abluminal layer. A cell impermeable layer is also disposed between the luminal layer and the abluminal layer. The cell impermeable layer is formed of an elastomeric material. The cell impermeable layer is impervious to cell migration across the layer when the stent graft is in a nominal state and in an expanded state. The stent graft is free of pleats or wrinkles when the stent graft is in the nominal state and in the expanded state.

### RELATED APPLICATIONS

This application claims priority to U.S. Provisional Application No. 63/269,366, filed on Mar. 15, 2022 and titled, “Stent Graft with Elastomeric Impermeable Layer,” which is hereby incorporated by reference in its entirety.

### TECHNICAL FIELD

The present disclosure relates generally to medical appliances. More particularly, the disclosure is related to stent grafts composed of two or more layers of material. In some embodiments, the disclosure is related to stent grafts composed of an elastomeric cell impermeable layer of material.

### BRIEF DESCRIPTION OF THE DRAWINGS

The embodiments disclosed herein will become more fully apparent from the following description and appended claims, taken in conjunction with the accompanying drawings. These drawings depict only typical embodiments, which will be described with additional specificity and detail through use of the accompanying figures in which:

FIG. 1A is a perspective cut-away view of a stent graft.

FIG. 1B is a cross-sectional view of the stent graft of FIG. 1A taken through line 1B-1B.

FIG. 1C is a cross-sectional view showing the layers of the stent graft of FIG. 1A.

FIG. 2 is a perspective view of a scaffolding of the stent graft of FIG. 1A.

FIG. 3A is a side cross-sectional view of a first layer of material of the stent graft of FIG. 1A disposed on a mandrel.

FIG. 3B is a side cross-sectional view of a scaffolding in a maximum expandable state disposed around the first layer of FIG. 3A.

FIG. 3C is a side cross-sectional view of the scaffolding of FIG. 3B radially compressed to a nominal state around the first layer of FIG. 3A.

FIG. 3D is a side cross-sectional view of a second layer of elastomeric material disposed around the first layer of FIG. 3A and the scaffolding of FIG. 3C.

FIG. 3E is a side cross-sectional view of a third layer of material disposed around the second layer of elastomeric material of FIG. 3D.

FIG. 4A is a side view of a delivery catheter disposed within a vessel of a patient at a lesion of a treatment site.

FIG. 4B is a side view of a stent graft coupled to the delivery catheter of FIG. 4A in a crimped state for deployment at the lesion of the treatment site.

FIG. 4C is a side view of the stent graft of FIG. 4B in a nominal state at the lesion of the treatment site.

FIG. 4D is a side view of the stent graft of FIG. 4B in an expanded state at the lesion of the treatment site.

FIG. 4E is a side view of the stent graft of FIG. 4B in a balloon expanded state at the lesion of the treatment site.

### DETAILED DESCRIPTION

Stent grafts may be deployed in various body lumens for a variety of purposes. Stent grafts may be deployed, for example, in the vascular system for a variety of therapeutic purposes, including the treatment of occlusions within the lumens of that system. The current disclosure may be applicable to stent grafts designed for the central venous (“CV”) system, peripheral vascular (“PV”) stents, abdominal aortic aneurysm (“AAA”) stents, bronchial stents, esophageal stents, biliary stents, coronary stents, gastrointestinal stents, neuro stents, thoracic aortic endographs, or any other stent or stent graft. Further, the present disclosure may be equally applicable to other prostheses such as stents, grafts, shunts, and so forth. Additionally, prostheses comprising a continuous lumen wherein a portion of the longitudinal length is reinforced, for example by a support structure, and a portion of the longitudinal length has no support structure are also within the scope of this disclosure. Any prosthesis composed of materials herein described may be configured for use or implantation within various areas of the body, including vascular, cranial, thoracic, pulmonary, esophageal, abdominal, or ocular application. Examples of prostheses within the scope of this disclosure include, but are not limited to, stents, vascular grafts, stent grafts, cardiovascular patches, reconstructive tissue patches, hernia patches, general surgical patches, heart valves, sutures, dental reconstructive tissues, medical device coverings and coatings, gastrointestinal devices, blood filters, artificial organs, ocular implants, and pulmonary devices, including pulmonary stents. For convenience, many of the specific examples included below reference stent grafts. Notwithstanding any of the particular stent grafts referenced in the examples or disclosure below, the disclosure and examples may apply analogously to any prosthesis.

As used herein, the term stent graft refers to a prosthesis configured for use within bodily structures, such as within body lumens. The stent graft may comprise a scaffolding or support structure, such as a frame, and/or a covering. In certain embodiments, the stent graft may be balloon expandable meaning the stent graft is radially expanded by an expandable balloon from a crimped state to an expanded state. In other embodiments, the stent graft may be self-expanding meaning that the stent graft is capable of expanding from a crimped state to an expanded state without application of an external force.

The components of the embodiments as generally described and illustrated in the Figures herein could be arranged and designed in a wide variety of different configurations. Thus, the following more detailed description of various embodiments, as represented in the Figures, is not intended to limit the scope of the disclosure, but is merely representative of various embodiments. While the various aspects of the embodiments are presented in drawings, the drawings are not necessarily drawn to scale unless specifically indicated.

The phrase “coupled to” refers to any form of interaction between two or more entities, including mechanical, electrical, magnetic, electromagnetic, fluid, and thermal interaction. Two components may be coupled to each other even though they are not in direct contact with each other. For example, two components may be coupled to each other through an intermediate component.

The directional terms “proximal” and “distal” are used herein to refer to opposite locations on a stent or another medical appliance. The proximal end of an appliance is defined as the end closest to the practitioner when the appliance is disposed within a deployment device that is being used by the practitioner. The distal end is the end opposite the proximal end, along the longitudinal direction of the appliance, or the end furthest from the practitioner. It is understood that, as used in the art, these terms may have different meanings once the appliance is deployed (i.e., the “proximal” end may refer to the end closest to the head or heart of the patient depending on application). For consistency, as used herein, the ends labeled “proximal” and “distal” remain the same regardless of whether the appliance is deployed.

The longitudinal direction of the stent graft is the direction along the axis of a generally tubular stent graft. In embodiments where the stent graft is composed of a scaffold structure coupled to one or more layers of a film or sheet-like component, such as a polymer layer, the support structure is referred to as the “scaffolding” or “frame,” and the polymer layer as the “covering” or “coating.” The terms “covering” and “coating” may refer to a single layer of polymer, multiple layers of the same polymer, or layers comprising distinct polymers used in combination. Furthermore, as used herein, the terms “covering” and “coating” refer only to a layer or layers that are coupled to a portion of the scaffolding; neither term requires that the entire scaffolding be “covered” or “coated.” In other words, medical appliances wherein a portion of the scaffolding may be covered and a portion may remain bare are within the scope of this disclosure. Finally, any disclosure recited in connection with coverings or coatings may analogously be applied to prostheses comprising one or more “covering” layers with no associated frame or other structure. For example, a hernia patch comprising any of the materials described herein as “coatings” or “coverings” is within the scope of this disclosure regardless of whether the patch further comprises a frame or other structure. Similarly, a tubular graft or shunt may be composed of the covering or layered materials recited herein, with no associated scaffolding.

Stent graft coverings within the scope of this disclosure may comprise multilayered constructs, composed of two or more layers that may be serially applied. Further, multilayered constructs may comprise nonhomogeneous layers, meaning adjacent layers have differing properties. Thus, as used herein, each layer of a multilayered construct may comprise a distinct layer, either due to the distinct application of the layers or due to differing properties between layers. As layers may be identified by their position, structure, or function, an individual layer may not necessarily comprise only a single material or a single microstructure.

Porous materials may be selectively permeable to various particles or biologic elements based on the pore sizes of the material. For example, materials with pore sizes smaller than 20 microns may be impermeable to cell types larger than 20 microns, such as foreign body giant cells. Similarly, materials with pore sizes smaller than eight microns may be impermeable to penetration by other cell types, such as red blood cells. In some embodiments, materials with pore sizes smaller than eight microns or smaller than six microns (including, for example, any value between zero and eight microns) may be impermeable to red blood cells.

As used herein, cellular impermeability does not require the complete exclusion of any cellular migration across a barrier. A material may be impermeable to red blood cell migration, for example, even if a small number of red bloods cells are able to cross the material. Accordingly, materials may be configured to substantially inhibit cellular migration across the material while meeting the definition of cellular impermeability. Constructs or layers where less than 0.1% of cells that contact the construct or layer will migrate across the layer may be termed cellular impermeable within the scope of this disclosure.

Moreover, constructs within the scope of this disclosure may be cell impermeable to any cell type, meaning that less than 0.1% of a particular cell type that contacts the construct (regardless of the cell type) will migrate across the construct wall. Similarly, constructs within the scope of this disclosure may be tissue impermeable, meaning that less than 0.1% of the mass or volume of tissue that contacts the construct will migrate across the construct wall.

Serially deposited fiber materials refers to materials composed at least partially of fibers successively deposited on a collector, a substrate, a base material, and/or previously deposited fibers. In some instances, the fibers may be randomly disposed, while in other embodiments the alignment or orientation of the fibers may be somewhat controlled or follow a general trend or pattern. Regardless of any pattern or degree of fiber alignment, because the fibers are deposited on the collector, substrate, base material, and/or previously deposited fibers, the fibers are not woven, but rather are serially deposited.

Rotational spinning is one example of how a material may be serially deposited as fibers. One embodiment of a rotational spinning process comprises loading a polymer solution or dispersion into a cup or spinneret configured with orifices on the outside circumference of the spinneret. The spinneret is then rotated, causing (through a combination of centrifugal and hydrostatic forces, for example) the flowable material within the spinneret to be expelled from the orifices. The material may then form a “jet” or “stream” extending from the orifice, with drag forces tending to cause the stream of material to elongate into a small diameter fiber. The fibers may then be deposited on a collection apparatus, a substrate, or other fibers. Once collected, the fibers may be dried, cooled, sintered, or otherwise processed to set the structure or otherwise harden the fiber mat. For example, polymeric fibers rotational spun from a dispersion may be sintered to remove solvents, fiberizing agents, or other materials as well as to set the structure of the mat. In one embodiment, for instance, an aqueous polytetrafluoroethylene (PTFE) dispersion may be mixed with polyethylene oxide (PEO) (as a fiberizing agent) and water (as a solvent for the PEO), and the mixture rotational spun. Sintering by heating the collected fibers may set the PTFE structure, evaporate off the water, and sublimate the PEO. Exemplary methods and systems for rotational spinning can be found in U.S. Pat. Application No. 13/742,025, filed on Jan. 15, 2013, and titled “Rotational Spun Material Covered Medical Appliances and Methods of Manufacture,” which is herein incorporated by reference in its entirety.

Rotational spinning may be utilized to create a variety of materials comprising serially deposited fibers. The microstructure or nanostructure of such materials, as well as the porosity, permeability, material composition, rigidity, fiber alignment, and so forth, may be controlled or configured to promote biocompatibility or influence interactions between the material and cells or other biologic material. A variety of materials may be serially deposited through processes such as rotational spinning: for example, polymers (e.g., PTFE), ceramics, metals, materials that may be melt-processed, or any other material having a soft or liquid form. A variety of materials may be serially deposited through rotational spinning while the material is in a solution, dispersion, molten or semi-molten form, and so forth. The present disclosure may be applicable to any material discussed herein being serially deposited as fibers onto any substrate or in any geometry discussed herein. Thus, examples of particular materials or structures given herein may be analogously applied to other materials and/or structures.

Expanded polytetrafluoroethylene (ePTFE) may also be used as a component of a layered prosthesis in some embodiments. ePTFE may be formed when a sheet of PTFE is heated and stretched. The sheet of ePTFE may be formed, for example, by extrusion or other methods. Heating and stretching of the PTFE sheet to form ePTFE changes the microstructure of the sheet, making it more porous and creating nodes of material with fibrils of material extending therebetween. U.S. Pat. No. 3,664,915 of W.L. Gore describes various processes for heating and stretching PTFE to create ePTFE. In some processes, the ePTFE will be expanded to a greater extent along a longitudinal direction as compared to a transverse direction. Thus, some ePTFE mats may be described as having an axis of expansion, or the direction in which the majority of the expansion was done. In some instances, the ratio of expansion in the longitudinal direction to expansion in a transverse direction may be between 10:1 and 20:1. In some embodiments, the majority of the fibrils of the ePTFE mat may be oriented, or substantially orientated along the axis of expansion.

Embodiments may be understood by reference to the drawings, wherein like parts are designated by like numerals throughout. It will be readily understood by one of ordinary skill in the art having the benefit of this disclosure that the components of the embodiments, as generally described and illustrated in the figures herein, could be arranged and designed in a wide variety of different configurations. Thus, the following more detailed description of various embodiments, as represented in the figures, is not intended to limit the scope of the disclosure, but is merely representative of various embodiments. While the various aspects of the embodiments are presented in drawings, the drawings are not necessarily drawn to scale unless specifically indicated.

Reference throughout this specification to “an embodiment” or “the embodiment” means that a particular feature, structure, or characteristic described in connection with that embodiment is included in at least one embodiment. Thus, the quoted phrases, or variations thereof, as recited throughout this specification are not necessarily all referring to the same embodiment.

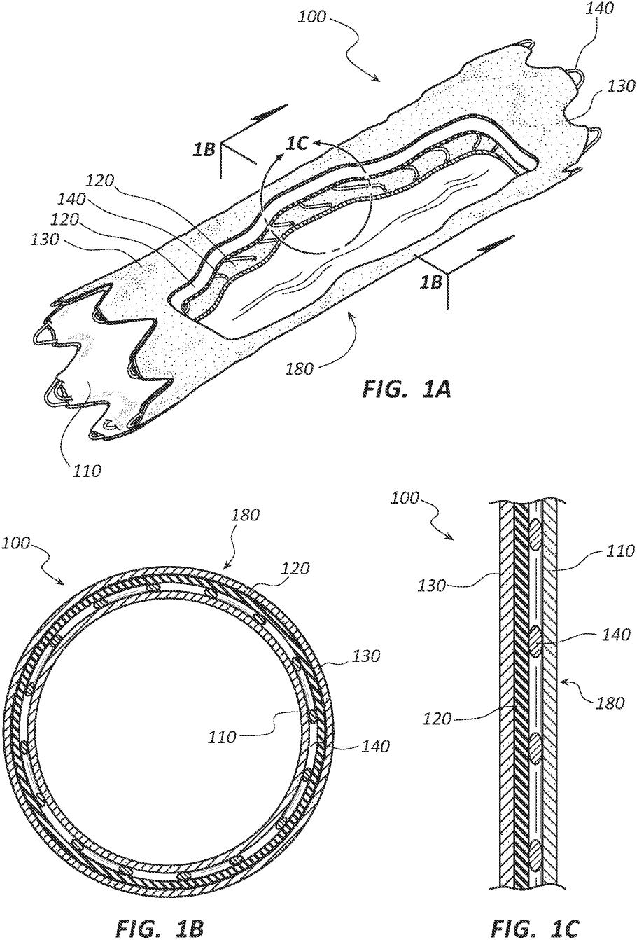

FIGS. 1A-1C illustrate an embodiment of a stent graft 100. Specifically, FIG. 1A is a perspective cut-away view of the stent graft 100. FIG. 1B is a cross-sectional view of the stent graft 100 taken through line 1B-1B of FIG. 1A. FIG. 1C is another cross-sectional view taken at 1C-1C showing the layers of the stent graft 100.

The stent graft 100 of FIGS. 1A-1C may be configured as a vascular stent graft. In the illustrated embodiment, a multilayered covering 180 of the stent graft 100 is shown with three distinct layers (a first layer 110, a second layer 120, and a third layer 130) disposed about a scaffolding or frame 140. In the illustrated embodiment, the covering 180 comprises a first layer 110 of rotationally spun PTFE. The rotationally spun PTFE first layer 110 defines a luminal surface of the stent graft 100. This luminal first layer 110 may be designed to interact with blood flowing within the vasculature. For example, the microporous structure of the rotationally spun first layer 110 may be configured to accommodate or allow endothelial cell growth on the luminal surface of the stent graft 100 when disposed within the vasculature of a patient. Alternatively or additionally, it is within the scope of this disclosure to use any serially deposited material on the luminal surface of the stent graft 100. For example, rotational spun PTFE, electrospun PTFE, or other polymers may be used on this layer.

The first layer 110 may comprise a wide variety of characteristics. For example, the first layer 110 with a percent porosity between 35% and 75%, including between 40% and 60%, and between 45% and 55%, is within the scope of this disclosure. Similarly, the first layer 110 with an average fiber diameter between 0.25 micron and 2.5 microns, including between 0.5 micron and 1.75 microns, and between 0.75 micron and 1.25 microns, is within the scope of this disclosure. Average pore diameter for the first layer 110 within the scope of this disclosure may range from one micron to five microns, including from two microns to four microns and from two microns to three microns. Finally, the average pore area of the first layer 110 within the scope of this disclosure may range from three square microns to 15 square microns, including from four square microns to 10 square microns, and from four square microns to eight square microns.

In the illustrated embodiment, the scaffolding 140 is disposed around the first layer 110 of the stent graft 100. This scaffolding 140 may comprise a metal stent—for example, a stent composed of nitinol, stainless steel, or alloys thereof. Additionally, other materials, such as polymer scaffolds, are within the scope of this disclosure. In some embodiments the scaffolding 140 may comprise a relative tight lattice, including a polymer lattice, which may tend to form a layer of the stent graft 100. References herein to a multilayered construct or multilayered component may be understood as referring to the entire stent graft 100, including the scaffolding 140, or may apply only to layers of material (such as 110, 120, and 130) disposed about the scaffolding 140.

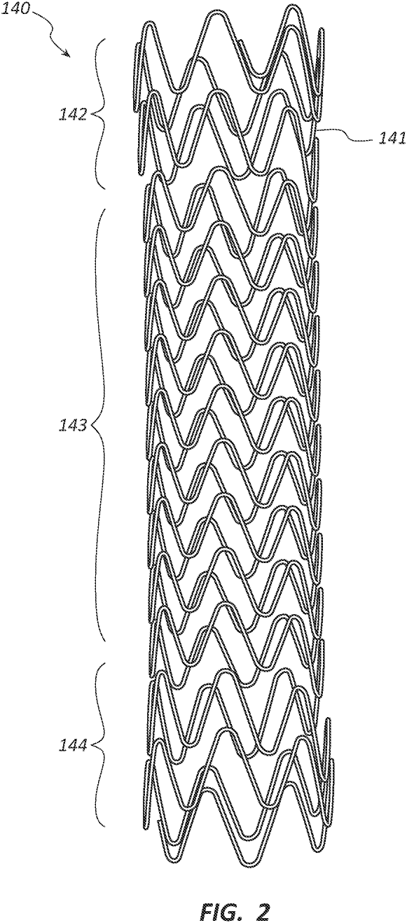

FIG. 2 is a perspective view of an embodiment of the scaffolding 140 for the stent graft 100. The scaffolding 140 may be coupled to coverings, including layered coverings, and may be configured to provide support and structure to the stent graft 100. For example, the scaffolding 140 may be configured to resist radial compression of the stent graft 100.

In some embodiments, the scaffolding 140 may be configured with different resistance to radial force along the longitudinal length of the scaffolding 140. For example, in the illustrated embodiment, the scaffolding 140 comprises a proximal portion 142, a mid-body portion 143, and a distal portion 144. The scaffolding 140 may be configured such that it provides greater resistance to radial compression in one or more of these portions 142, 143, 144 as compared to at least one other portion 142, 143, 144 of the scaffolding 140. Differing resistance to radial force along the length of the scaffolding 140 may be designed to provide strength in certain areas (such as an area to be treated, such as an aneurysm) while providing softer portions of the scaffolding 140 that may allow the scaffolding 140 to interact with healthy portions of the body in a more atraumatic way. Thus, one or more portions of the scaffolding 140 may be configured to hold open diseased tissue within a body lumen.

The radial resistance of the scaffolding 140 may be a result of the material used to create the scaffolding 140; variations in the structure of the scaffolding 140, such as the degree to which the scaffolding 140 comprises a more open or more closed design; and other design parameters. In some embodiments within the scope of this disclosure, the radial force along portions of the same scaffolding 140 may vary by between 10% and 30%, between 30% and 60%, between 60% and 100%, more than 100%, and more than 140%.

In some embodiments, the scaffolding 140 may be configured such that the resistance to radial compression of the scaffolding 140 is greater in the mid-body portion 143 of the scaffolding 140 as compared to the proximal 142 and/or distal 144 portions thereof. In other embodiments the mid-body portion 143 may have less resistance to radial compression or, in other words, may be softer than the proximal 142 and/or distal 144 portions thereof. Still further, in some embodiments the proximal 142 and/or distal 144 portions of a scaffolding 140 may be configured to reduce tissue aggravation at the edge of the scaffolding 140. In some instances, the resistance to radial compression of one or more ends of the scaffolding 140 may be configured to reduce the occurrence of edge stenosis. Moreover, the resistance to radial compression of one or more ends of the scaffolding 140 may be configured to promote endothelial cell growth on a surface of a stent graft 100 coupled to the scaffolding 140. The resistance to radial compression along one or more portions of the scaffolding 140 may be configured to match the compliance of a body vessel in which the scaffolding 140 is designed for deployment.

In certain embodiments, the scaffolding 140 may comprise metals, including stainless steel, nitinol, various super elastic or shape memory alloys, and so forth. In other embodiments, the scaffolding 140 may comprise polymers. Further, the scaffolding 140 may comprise one or more biologic agents, including embodiments wherein a metal or polymeric scaffolding is integrated with a drug or other biologic agent.

The scaffolding 140 may be formed in a variety of ways. In some embodiments, the scaffolding 140 may be formed of a wire 141. Further, the scaffolding 140 may be formed from a tube of material, including embodiments wherein the scaffolding 140 is cut from a tube of material. The scaffolding 140 may be formed using laser cutting, etching processes, and powdered metallurgy and sintering processes; formed from molds; and formed using rapid manufacturing techniques.

The stent graft 100, with or without the scaffolding 140, may be configured to exert an outward radial force when disposed within a body lumen. This force may be configured to keep the lumen open, prevent restenosis, inhibit migration of the stent or stent graft, and so forth. However, the stent graft 100 that subjects body lumens to high radial forces may provoke an unwanted biologic response and/or result in unnecessary trauma to the body lumen. Accordingly, the stent graft 100 may be configured to exert a radial force within a range that is acceptable for healing and trauma, while still achieving treatment goals.

In some embodiments, the stent graft 100 may be configured such that the stent graft 100 resists localized compression, for example due to a point or pinch force, even when the localized force exceeds the circumferential outward radial force of the stent graft 100. In other words, the stent graft 100 may be configured to resist relatively high point forces (for example, as may be exerted by a ligament or other biologic structure) on the stent graft 100 without exerting high radial forces on the entire body lumen.

The second layer 120 of the stent graft 100 may be cell impermeable. For example, in the illustrated embodiment of FIGS. 1A-1C, the second layer 120 may comprise an elastomeric polymer layer that is cell impermeable. The impermeable second layer 120 may be configured to prevent cellular migration across the stent graft 100. Containment of cellular migration across the stent graft 100 may lengthen the useful life of the stent graft 100, as bodily tissues are prevented from growing through the stent graft 100 and occluding the lumen thereof.

In some embodiments, the impermeable second layer 120 may comprise an elastomeric polymer material having a high elastic characteristic. For example, the elastomeric polymer material may be silicone, thermoplastic elastomer, polyurethane, fluoroelastomers, or thermoplastic polyolefin. Other elastomeric polymer materials are contemplated within the scope of this disclosure. In certain embodiments, the elastomeric polymer material may be sprayed, dipped, or laminated onto the first layer 110 and/or the scaffolding 140. In other embodiments, the elastomeric polymer material within the scope of this disclosure may thus be applied as a film or membrane that is rolled onto or otherwise applied to the first layer 110 and/or the scaffolding 140.

In some embodiments, the second layer 120 and the third layer 130 may be constructed as a composite layer. For instance, a third layer 130 comprising ePTFE may be sprayed or dipped with the elastomeric polymer material such that the elastomeric polymer material coats the ePTFE and fills in the pores and openings in the ePTFE. A composite layer of ePTFE and elastomeric polymer material may thus be configured with the properties and functions of both the second layer 120 and the third layer 130.

In the illustrated embodiment, the elastomeric polymer material of the second layer 120 may be circumferentially stretched when the stent graft 100 is radially expanded from a nominal state to an expanded state. The nominal state may be defined within the scope of this disclosure as a state where the stent graft 100 is neither radially compressed nor radially expanded. A diameter of the stent graft 100 in the nominal state may range from about 3 millimeters to about 55 millimeters and a diameter of the stent graft 100 in the expanded state may range from about 0% to about 200% larger than the nominal state diameter. In the nominal state the second layer 120 is not circumferentially stretched and in the expanded state the second layer 120 may be circumferentially stretched from about 0% to about 200%. For example, when the stent graft 100 is in the nominal state, a diameter of the stent graft 100 may be about eight millimeters having a circumference of about 25.12 millimeters. When the stent graft 100 is in the expanded state, the diameter of the stent graft 100 may range from about eight millimeters to about 16 millimeters having a circumference ranging from about 25.12 millimeters to about 50.24 millimeters. Thus, the circumference of the stent graft 100 can be from 0% to 100% larger when the stent graft 100 is in the expanded state than when in the nominal state.

In some embodiments, the elastomeric polymer material of the second layer 120 can be cell impermeable when the stent graft 100 is in the nominal state and in the expanded state. In other words, the elastomeric polymer material can substantially prevent migration of cells across the second layer 120 when the elastomeric material is not stretched and when it is stretched up to 100%.

The scaffolding 140 can be configured to provide a radially outward directed force capable of overcoming an elastic radial expansion resistance force applied by the elastomeric polymer material of the second layer 120.

The stent graft 100 may further comprise a third layer 130 disposed around the impermeable second layer 120. This third layer 130 may define an abluminal surface of the stent graft 100. In some instances, the third layer 130 may comprise ePTFE and may be densified and/or have a relatively small inter-nodal distance (IND) or pore size. The third layer 130 may be configured to provide strength to the stent graft 100.

Any of the layers discussed above (110, 120, 130) may be composed of one or more sublayers. For example, if the first layer 110 is composed of serially deposited PTFE fibers, the first layer 110 may, in turn, comprise multiple sublayers of serially deposited PTFE fibers. In a certain embodiment, the first layer 110 may thus consist of a first sublayer comprising serially deposited PTFE fibers and a second sublayer also comprising serially deposited PTFE fibers. The sublayers may be deposited at different times during manufacture and/or may be sintered separately, for example. The first layer 110 may also include other materials, disposed between the sublayers, for example to aid in coupling the sublayers to each other. Any number of sublayers may be combined within a single layer.

In some embodiments, a wall thickness of the covering 180 for the stent graft 100 may be between 50 microns and 500 microns, including between 50 microns and 450 microns, between 50 microns and 400 microns, between 50 microns and 350 microns, between 50 microns and 300 microns, between 50 microns and 250 microns, between 50 microns and 140 microns, between 50 microns and 150 microns, and between 75 microns and 125 microns.

The wall thickness of any individual layer (110, 120, 130) and the scaffolding 140 may be between five microns and 100 microns, including between five microns and 75 microns, between five microns and 60 microns, between 25 microns and 75 microns, between 10 microns and 30 microns, and between five microns and 15 microns. Any layer described herein may fall within any of these ranges, and the thicknesses of each layer (110, 120, 130) and the scaffolding 140 may be configured such that the total wall thickness of the covering 180 falls within the ranges described above.

Multilayered constructs that have more or fewer layers than the stent graft 100 are likewise within the scope of this disclosure. For example, constructs having two, three, four, five, six, seven, or more layers are all within the scope of this disclosure.

In certain embodiments, the layers 110, 120, 130 are free of pleats or wrinkles when the stent graft 100 is in the nominal state and/or the expanded state. Pleats or wrinkles can form from excess circumferential material when the stent graft 100 is radially compressed or crimped to a diameter smaller than a build or nominal diameter as will be described below. The nominal diameter is defined as the diameter of the stent graft 100 when the layers 110, 120, 130 are not radially compressed or radially expanded. This configuration can increase the potential for laminar blood flow along the luminal surface of the first layer 110 and can reduce the incidence for thrombus formation and occlusion of the stent graft 100.

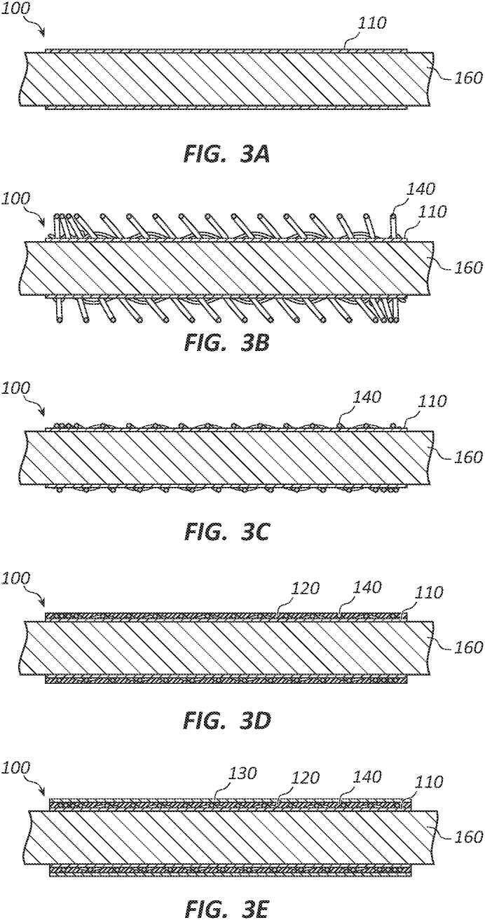

With reference to the stent graft 100 of FIGS. 1A-1C, a method of manufacture, as illustrated in FIGS. 3A-3E, may comprise serially depositing PTFE fibers on a mandrel 160 or other collection surface and sintering the fibers as illustrated in FIG. 3A. This layer of serially deposited PTFE fibers may form the first layer 110 of the stent graft 100. The mandrel 160 may have a diameter ranging from about two millimeters to about 55 millimeters. The diameter of the mandrel 160 may be substantially equivalent to the nominal diameter of the stent graft 100.

The scaffolding 140 may be applied around the first layer 110 of the stent graft 100 as shown in FIG. 3B. Prior to applying the scaffolding 140 around the first layer 110, the scaffolding 140 may be formed on a separate mandrel with a diameter equivalent to a maximum expandable diameter of the scaffolding 140. The maximum expandable diameter can be equivalent to or greater than a maximum diameter of a vessel the stent graft 100 is intended to be used in for treatment. For example, the maximum expandable diameter of the scaffolding 140 may be 17 millimeters and the maximum vessel diameter the stent graft 100 intended to be used may be 16 millimeters. Following forming of the scaffolding 140 at the maximum expandable diameter, the scaffolding 140 can be configured (e.g., heat set) such that the scaffolding 140 may return to the maximum expandable diameter following crimping and deployment within the vessel.

As illustrated in FIG. 3C, the scaffolding 140 can be radially compressed around the first layer 110 such that the diameter of the scaffolding 140 is substantially equivalent to the diameter of the mandrel 160 or the nominal diameter of the stent graft 100.

The second layer 120 may be applied to the first layer 110 and/or the scaffolding 140 as illustrated in FIG. 3D. The second layer 120 may comprise the elastomeric polymer material. The elastomeric polymer material may be applied using any suitable technique, such as spraying, dipping, laminating, or disposing an extruded tube of the elastomeric polymer material over the first layer 110 and/or the scaffolding 140. In other embodiments, the elastomeric polymer material may be applied as a film or membrane. In still other embodiments, the elastomeric material may be applied using a reflow technique. Additionally, in some embodiments the second layer 120 may be applied around the first layer 110 before the first layer 110 is sintered. Sintering of the first layer 110 while the second layer 120 is disposed therearound may facilitate bonding between the first 110 and second 120 layers.

As illustrated in FIG. 3E, the third layer 130 comprising ePTFE may be disposed over or around the second layer 120. The third layer 130 may be sintered after it is applied around the second layer 120. The constructed stent graft 100 may be removed from the mandrel 160. When removed from the mandrel 160, the stent graft 100 may have the nominal diameter of the mandrel 160, for example eight millimeters, and be free of pleats or wrinkles, as the scaffolding 140, initially formed at a larger diameter, may tend to apply a radial outwardly directed force on the construct cover to eliminate or reduce wrinkles. The diameter of the layers 110, 120, 130 and the scaffolding 140 may be substantially equivalent to the nominal diameter of the stent graft 100.

In some embodiments, mechanical properties of the third layer 130 may be derived from the relative disposition of ePTFE sublayers that comprise the overall ePTFE third layer 130. Each sublayer may impart different properties to the overall construct. For example, the sublayers may be stronger in the direction the ePTFE was expanded than in a transverse direction. Application of such sublayers such that the axis of expansion of each sublayer is perpendicular to the axis of expansion of adjacent layers may create a layer of ePTFE with more uniform longitudinal and radial properties. Constructs within the scope of this disclosure may comprise ePTFE sublayers disposed such that the axis of expansion of a first sublayer is disposed at any angle to the axis of expansion of an adjacent sublayer. Further, constructs wherein the axis of expansion of an ePTFE sublayer is aligned with the central axis of the stent graft 100, for example the longitudinal axis of the stent graft 100, are within the scope of this disclosure.

Furthermore, embodiments wherein the axis of expansion of an ePTFE sublayer is disposed at an angle to the central axis of the stent graft 100, for example the longitudinal axis of the stent graft 100, are also within the scope of this disclosure. For example, embodiments wherein one or more ePTFE layers are oriented such that the axis of expansion is disposed between 0° and 25° to the center axis of the stent graft 100, including angles between 0° and 15° are within the scope of this disclosure. For example, the covering 180 of the stent graft 100 may comprise one or more layers (110, 120, 130) wherein one or more layers or sublayers formed of ePTFE are disposed as discussed above. For example, in some embodiments the third layer 130 may be comprised of ePTFE (whether constructed as a composite layer with the second layer 120 or constructed separately) and the axis of expansion of the ePTFE disposed with respect to a center axis of the stent along the ranges of angles discussed above. In some embodiments, this orientation may impart additional elasticity to the covering 180, and may be configured to allow the covering to be dilated or stretched beyond a nominal diameter without rupturing or tearing the layers of the covering 180. In some embodiments, stent grafts 100 so constructed by may be expandable 150%, 200%, or more past a nominal diameter without tearing or rupturing. For example, in some embodiments, a stent graft 100 comprising a cover 180 with a nominal diameter of 8 mm may be expandable to 12 mm or more without tearing layers or comprising the integrity of one or more impermeable layers. In some such embodiments, the impermeable layers may have sufficient elasticity (including silicone layers, FEP layers, and so forth) to allow for such expansion.

In some embodiments sublayers of ePTFE may be constructed at different angles. For example, a layer of ePTFE may be constructed by sublayers by obtaining a strip of ePTFE that is narrower than the length of the stent graft 100. In some such instances, the sublayers may be disposed to impart different strengths in differing directions. For example, the initial strip may be helically wrapped around the first layer 110 of the stent graft 100. In some instances, the strip may be wrapped at about 45° to the longitudinal axis of the stent graft 100. The strip may comprise a sublayer of the third layer 130. A second sublayer may be applied, also at about 45° to the longitudinal axis of the stent graft 100, but applied such that the axis of expansion of the second sublayer is perpendicular to the first strip applied. The combined strength of the sublayers may thus be arranged such that the sum of the strength of the sublayers is similar in the longitudinal and radial directions of the stent graft 100. Any other angle of relative positioning of sublayers is within the scope of this disclosure, and the relative angles may be configured to create a construct with certain properties and strengths in various directions.

Methods of deploying the stent graft 100 within the body at a treatment site are also within the scope of this disclosure. Similarly, methods of resisting transmural tissue growth or migration across the stent graft 100 when the stent graft 100 is in the expanded state are within the scope of this disclosure. For example, deployment of the stent graft 100 having a blood-contacting layer configured to promote endothelial growth and at least one impermeable layer configured to resist cell migration through the impermeable layer would be related to such methods.

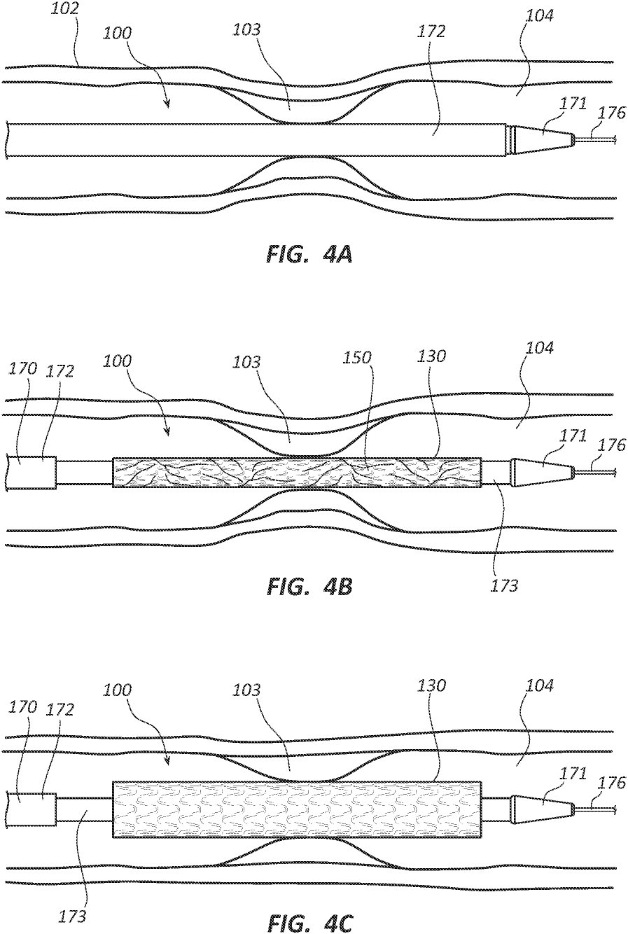

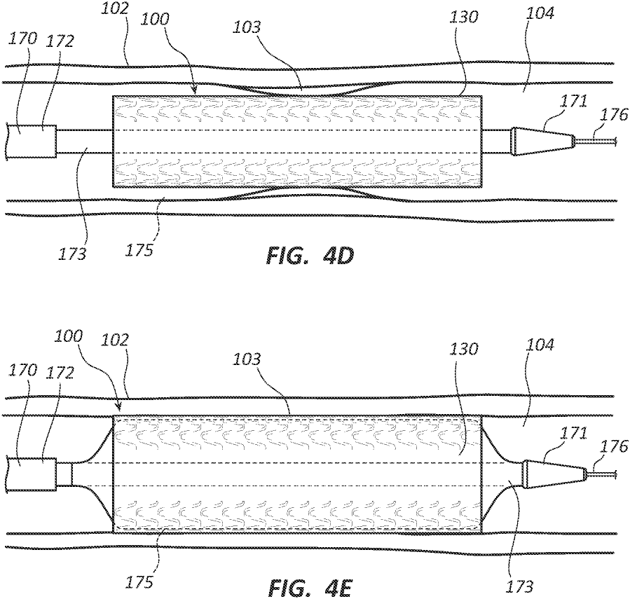

FIGS. 4A-4E illustrate a method of deploying the stent graft 100 at a treatment site having a lesion within a lumen of a vessel. As illustrated in FIG. 4A, a stent graft delivery catheter 170 having a distal tip 171 is disposed over a guidewire 176 through a lesion 103 (e.g., narrowing of a vessel) within a lumen 104 of a vessel 102.

As illustrated in FIG. 4B, an outer sheath 172 of the delivery catheter 170 can be retracted to expose the stent graft 100 in a crimped state around an inner sheath 173 of the delivery catheter 170. As shown, in the crimped state the stent graft 100 includes pleats or wrinkles 150 of any one or all of the layers 110, 120, 130.

As illustrated in FIG. 4C, the stent graft 100 may self-expand to the nominal state having the nominal diameter and apply a radial outward force to the lesion 103 to expand a diameter of the lesion 103. For example, the stent graft may self-expand to a nominal diameter of eight millimeters. In other embodiments, the stent graft 100 may be balloon expanded to the nominal diameter. As shown, in the nominal state any one or all of the layers 110, 120, 130 are free of pleats or wrinkles and the second layer 120 is cell impermeable.

As illustrated in FIG. 4D, the stent graft 100 may self-expand to an expanded diameter ranging between the nominal diameter and the maximum expandable diameter to further increase the diameter of the lesion 103. For example, the stent graft 100 may self-expand between a nominal diameter of eight millimeters to a maximum expandable diameter of 16 millimeters. In some embodiments, the stent graft 100 may self-expand between a diameter ranging from about two millimeters to about 55 millimeters. In another embodiment, as illustrated in FIG. 4E, the stent graft 100 may be balloon expanded by a balloon 175 of the delivery catheter 170 or a separate balloon catheter to the diameter between the nominal diameter and the maximum expandable diameter. As depicted in FIGS. 4D and 4E, any one or all of the layers 110, 120, 130 are free of pleats or wrinkles. The second layer 120 may be circumferentially stretched from about 0% to about 200% while maintaining cell impermeability.

Thus, depending on application, vessel size, use of balloon expanders, and so forth, the stent graft 100 may expand to a diameter between the nominal diameter of the construct, corresponding to the diameter of the mandrel 160 on which the construct is formed, and the maximum expandable diameter of the scaffolding 140. The layers 110, 120, 130 may be configured to stretch between these two sizes and maintain characteristics such as cell impermeability over this range.

Any methods disclosed herein comprise one or more steps or actions for performing the described method. The method steps and/or actions may be interchanged with one another. In other words, unless a specific order of steps or actions is required for proper operation of the embodiment, the order and/or use of specific steps and/or actions may be modified. For example, a method of treating a vascular lesion may include one or more of the following steps: positioning a vascular prosthesis adjacent a vascular lesion; radially expanding the vascular prosthesis to apply a radially outward oriented force to the vascular lesion; and circumferentially stretching an elastomeric layer of the vascular prosthesis from a nominal state to an expanded state, wherein the vascular prosthesis is free of pleats when the vascular prosthesis is nominal state and the expanded state, and wherein the elastomeric layer is cell impermeable when the vascular prosthesis is in the expanded state. Other steps are also contemplated.

In the above description of embodiments, various features are sometimes grouped together in a single embodiment, figure, or description thereof for the purpose of streamlining the disclosure. This method of disclosure, however, is not to be interpreted as reflecting an intention that any claim requires more features than those expressly recited in that claim. Rather, as the following claims reflect, inventive aspects lie in a combination of fewer than all features of any single foregoing disclosed embodiment.

The phrase “coupled to” refers to any form of interaction between two or more entities, including mechanical, electrical, magnetic, electromagnetic, fluid, and thermal interaction. Two components may be coupled to each other even though they are not in direct contact with each other. For example, two components may be coupled to each other through an intermediate component.

References to approximations are made throughout this specification, such as by use of the terms “substantially” or “about.” For each such reference, it is to be understood that, in some embodiments, the value, feature, or characteristic may be specified without approximation. For example, where qualifiers such as “about” and “substantially” are used, these terms include within their scope the qualified words in the absence of their qualifiers. For example, where the term “substantially prevent” is recited with respect to a feature, it is understood that in further embodiments, the feature can have a precisely prevention configuration.

The terms “a” and “an” can be described as one, but not limited to one. For example, although the disclosure may recite a housing having “a stopper,” the disclosure also contemplates that the housing can have two or more stoppers.

Unless otherwise stated, all ranges include both endpoints and all numbers between the endpoints.

Recitation in the claims of the term “first” with respect to a feature or element does not necessarily imply the existence of a second or additional such feature or element.

The claims following this written disclosure are hereby expressly incorporated into the present written disclosure, with each claim standing on its own as a separate embodiment. This disclosure includes all permutations of the independent claims with their dependent claims. Moreover, additional embodiments capable of derivation from the independent and dependent claims that follow are also expressly incorporated into the present written description.

Without further elaboration, it is believed that one skilled in the art can use the preceding description to utilize the invention to its fullest extent. The claims and embodiments disclosed herein are to be construed as merely illustrative and exemplary, and not a limitation of the scope of the present disclosure in any way. It will be apparent to those having ordinary skill in the art, with the aid of the present disclosure, that changes may be made to the details of the above-described embodiments without departing from the underlying principles of the disclosure herein. In other words, various modifications and improvements of the embodiments specifically disclosed in the description above are within the scope of the appended claims. Moreover, the order of the steps or actions of the methods disclosed herein may be changed by those skilled in the art without departing from the scope of the present disclosure. In other words, unless a specific order of steps or actions is required for proper operation of the embodiment, the order or use of specific steps or actions may be modified. The scope of the invention is therefore defined by the following claims and their equivalents

1. A multilayered vascular prosthesis, comprising:
a first layer comprising serially deposited polytetrafluoroethylene (PTFE) fibers providing a luminal surface of a vascular prosthesis;
a second layer comprising an elastomeric material;
a third layer comprising expanded polytetrafluoroethylene (ePTFE) providing an abluminal surface of the vascular prosthesis, wherein the second layer is disposed between the first layer and the third layer; and
a scaffolding disposed between the first layer and the second layer;
wherein the vascular prosthesis has a nominal diameter when in a nominal state, wherein the second layer is non-stretched;
wherein the vascular prosthesis has an expanded diameter when in an expanded state, wherein the expanded diameter is larger than the nominal diameter; and
wherein the vascular prosthesis is free of pleats when the vascular prosthesis is in the nominal state and the expanded state.

2. The multilayered vascular prosthesis of claim 1, wherein the second layer is cell impermeable when the vascular prosthesis is in the nominal state and the expanded state.

3. The multilayered vascular prosthesis of claim 1, wherein the elastomeric material comprises any one of silicone, thermoplastic elastomer, polyurethane, fluoroelastomer, thermoplastic polyolefin, or any combination thereof.

4. The multilayered vascular prosthesis of claim 1, wherein the axis of expansion of one or more sublayers of the third layer of ePTFE is disposed at an angle of between 0° and 25° to a longitudinal axis of the multilayered vascular prosthesis.

5. The multilayered vascular prosthesis of claim 1,
wherein the nominal diameter ranges from two millimeters to 55 millimeters, and
wherein the expanded diameter ranges from four millimeters to 55 millimeters.

6. The multilayered vascular prosthesis of claim 1, wherein the second layer circumferentially stretches when the vascular prosthesis is expanded from the nominal state to the expanded state, and wherein the circumferential stretch ranges from 0% to 200%.

7. The multilayered vascular prosthesis of claim 1, wherein each of the first layer and the third layer circumferentially stretch when the vascular prosthesis is expanded from the nominal state to the expanded state, and wherein the circumferential stretch ranges from 0% to 200%.

8. The multilayered vascular prosthesis of claim 1, wherein the scaffolding is configured to resist a radially inward oriented force applied by the second layer to prevent the vascular prosthesis from contracting from the expanded state to the nominal state.

9. The multilayered vascular prosthesis of claim 1, wherein the scaffolding is configured to apply a radially outward oriented force to circumferentially stretch the second layer 100%.

10. The multilayered vascular prosthesis of claim 1, wherein the serially deposited PTFE fibers are rotational spun.

11. The multilayered vascular prosthesis of claim 1, wherein the vascular prosthesis is a self-expanding stent graft.

12. The multilayered vascular prosthesis of claim 1, wherein the vascular prosthesis is a balloon expandable stent graft.

13. A method of constructing a multilayered vascular prosthesis, the method comprising:
serially depositing polytetrafluoroethylene (PTFE) fibers on a mandrel to form a first layer, wherein the first layer comprises a nominal diameter;
forming a scaffolding, wherein the scaffolding comprises a maximum diameter;
disposing the scaffolding over the first layer;
radially compressing the scaffolding, wherein the radially compressed scaffolding comprises the nominal diameter;
disposing an elastomeric material over the first layer and the structural member to form a second layer; and
disposing a third layer comprising an expanded polytetrafluoroethylene (ePTFE) material over the second layer.

14. The method of claim 13, wherein serially depositing the PTFE fibers on the mandrel comprises rotationally spinning a PTFE solution.

15. The method of claim 13, wherein disposing the elastomeric material over the first layer and the structural member comprises any one of spraying, dipping, painting, laminating, and disposing an extruded tube of the elastomeric material over the first layer and the structural member.

16. The method of claim 13, wherein the first, second, and third layers are free of pleats.

17. The method of claim 13,
wherein the nominal diameter ranges from two millimeters to 55 millimeters, and
wherein the maximum diameter is 200% larger than the nominal diameter.

18. A multilayered vascular prosthesis, comprising:
a first layer comprising polytetrafluoroethylene (PTFE) providing a luminal surface of a vascular prosthesis;
a second layer comprising an elastomeric material;
a third layer comprising polytetrafluoroethylene (ePTFE) providing an abluminal surface of the vascular prosthesis, wherein the second layer is disposed between the first layer and the third layer; and
a scaffolding disposed between the first layer and the second layer;
wherein the vascular prosthesis has a nominal diameter when in a nominal state, wherein the second layer is non-stretched;
wherein the vascular prosthesis has an expanded diameter when in an expanded state, wherein the expanded diameter is larger than the nominal diameter; and
wherein the vascular prosthesis is free of pleats when the vascular prosthesis is in the nominal state and the expanded state.

19. The multilayered vascular prosthesis of claim 18, wherein the second layer is cell impermeable when the vascular prosthesis is in the nominal state and the expanded state.

20. The multilayered vascular prosthesis of claim 18, wherein an axis of expansion of one or more sublayers of an ePTFE layer is disposed at an angle of between 0° and 25° to a longitudinal axis of the multilayered vascular prosthesis.
